(19) United States
(12) Patent Application Publication
(10) Pub. No.: US 2017/0275335 A1
(43) Pub. Date: Sep. 28, 2017

(54) IMPROVED PROCESS FOR PREPARATION OF AMORPHOUS LINACLOTIDE

(71) Applicant: DR. REDDY'S LABORATORIES LIMITED, Hyderabad, TS (IN)

(72) Inventors: Dipak KALITA, Hyderabad (IN); Ramrao Jogdand NIVRUTTI, Hyderabad (IN); Kesavan BALAKUMARAN, Hyderabad (IN); Shivshankar DESHMUKH, Hyderabad (IN); Naga Chandra Sekhar VUTUKURU, Hyderabad (IN); Vara Prasad KASINA, Hyderabad (IN); Sivannarayana NALAMOTHU, Hyderabad (IN); Mohan Sundaram VILVA, Kakinada (IN); Rashid Abdul Rehman KHAN, Hyderabad (IN); Ramreddy TIRUMALAREDDY, Hyderabad (IN); Sairam MUSTOORI, Hyderabad (IN)

(21) Appl. No.: 15/325,134

(22) PCT Filed: Jul. 21, 2015
(86) PCT No.: PCT/IB2015/055513

§ 371 Date: Jan. 10, 2017

(30) Foreign Application Priority Data
Jul. 22, 2014 (IN) 3587/CHE/2014

(51) Int. Cl. C07K 7/08; C07K 1/02; C07K 1/113; C07K 1/18; C07K 1/20

(52) U.S. Cl. CPC C07K 7/08; C07K 1/18; C07K 1/20; C07K 1/1133; C07K 1/02

(57) ABSTRACT

The present application relates to an improved process for the formation of disulfide bonds in linaclotide. The present application also relates to an improved process for the purification of linaclotide.

### FIELD OF INVENTION

The present application relates to an improved process for the preparation of amorphous linaclotide. Specifically, the present application relates to an improved process for the formation of disulfide bonds in linaclotide. The present application further relates to a purification process for the preparation of amorphous linaclotide.

### Introduction

Linaclotide is a 14-residue peptide which is an agonist of the guanylate cyclase type-C receptor. Linaclotide may be used for the treatment of chronic constipation and irritable bowel syndrome. Structurally, linaclotide has three disulfide bonds and they are present between Cys¹-Cys⁶, Cys²-Cys-¹⁰ and Cys⁵-Cys¹³. The structure of linaclotide is shown below:

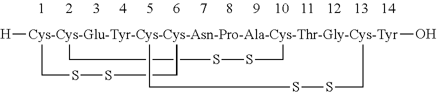

Benitez et al. Peptide Science, 2010, Vol. 96, No. 1, 69-80 discloses a process for the preparation of linaclotide. The process involves the use of 2-chlorotrityl (CTC) resin and 9-fluorenylmethoxycarbonyl (Fmoc) chemistry. The Cys residues are protected by Trt (trityl) group. The amino acids are coupled to one another using 3 equivalents of 1-[bis(dimethylamino)methylene]-6-chloro-1H-benzotriazolium hexafluorophosphate 3-oxide (HCTU) as coupling agent and 6 equivalents of diisoprpylethylamine (DIEA) as base in dimethylformamide (DMF). The Fmoc group is removed using piperidine-DMF (1:4). The Cys residues are incorporated using 3 equivalents of N,N′-diisopropylcarbodiimide (DIPCDI) as coupling agent and 3 equivalents of 1-hydroxybenzotriazole (HOBt) as an activating agent. After the elongation of the peptide chain, the peptide was cleaved from the solid support (CTC resin) by first treating with 1% trifluoroacetic acid (TFA) and then with a mixture of TFA, triisoprpylsilane (TIS) and water in the ratio of 95:2.5:2.5. The disulfide bonds are prepared by subjecting the linear peptide to air oxidation in sodium dihydrogen phosphate (100 mM) and guanidine hydrochloride buffer (2 mM).

US2010/261877A1 discloses a process for purification of linaclotide. The process involves first purification of crude peptide by reverse-phase chromatographic purification followed by concentrating the purified pools and dissolving the purified linaclotide in aqueous-isopropanol or aqueous-ethanol and spray-drying the solution to afford pure Linaclotide.

The synthesis of a peptide containing disulfide bridges is difficult for two main reasons; one is potential risk of racemization during the formation of linear chain and the other is mis-folding of the disulfide bridges. Hence, there is a need in the art to a cost-effective process for the preparation of pure linaclotide.

### SUMMARY

One aspect of the present application relates to an improved process for the preparation of amorphous linaclotide.

Another aspect of the present application relates to processes for preparing disulfide bridges of linaclotide.

Yet another embodiment of the present application relates to a purification process for preparing amorphous linaclotide.

### BRIEF DESCRIPTION OF THE DRAWING

FIG. 1 shows a powder X-ray diffraction (PXRD) pattern of amorphous linaclotide.

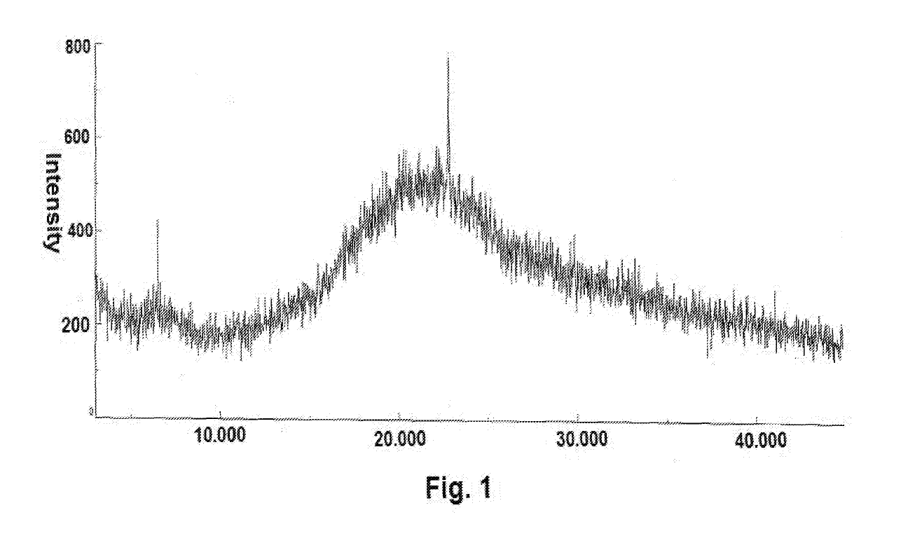

### DETAILED DESCRIPTION

The present application relates to an improved process for the preparation of amorphous linaclotide.

The present application also relates to a process for preparing disulfide bridges of linaclotide. Specifically, the present application relates to a process for preparing crude linaclotide by treating a linear chain of peptide of formula (I) with a suitable reagent to prepare appropriate disulfide bridges within linear chain of peptide of formula (I)

Formula (I)
1 2 3 4 5 6 7 8 9 10 11 12 13 14
H-Cys-Cys-Glu-Tyr-Cys-Cys-Asn-Pro-Ala-Cys-Thr-Gly-Cys-Tyr-OH

wherein, the suitable reagent is selected from the group consisting of polymer bound complex of sulfur trioxide-pyridine, dimethyl sulfoxide (DMSO) in water, a complex of pyridine-sulfur trioxide, guanidine hydrochloride, clear-OX™, reduced glutathione, air in presence of DMSO, solid supported (2,2,6,6-tetramethylpiperidinyl-1-yl)oxy (TEMPO) in presence of a co-oxidant, in water without any oxidant, hydrogen peroxide, potassium ferricyanide, manganese oxide, montmorillonite K-10, trimethylamine sulfur trioxide, vanadium pentoxide and cysteine-cystine.

The present application also relates to a purification of crude linaclotide obtained by the processes described above, to provide amorphous form of linaclotide.

First aspect of the present application relates to a process for preparing linaclotide by treating linear chain of peptide of formula (I) with polymer bound complex of sulfur trioxide-pyridine. Polymer bound complex of sulfur trioxide-pyridine is available commercially. Specifically, polyvinyl polymer bound complex of sulfur trioxide-pyridine may be used for the preparation of crude linaclotide. The reaction between linear chain of peptide of formula (I) and polyvinyl polymer bound sulfur trioxide may be performed in an aqueous solution in basic pH. Specifically, the reaction may be performed in water at a pH from about 8.0 to about 10.0. The reaction may be performed at about 15° C. to about 50° C. for about 1 hour to about 48 hours. Specifically, the reaction may be performed at about 20° C. to about 30° C. for about 15 hours to about 30 hours. The reaction may be worked-up by acidification of the reaction medium by a suitable acid such as trifluoroacetic acid, acetic acid and the product may be isolated by any known methods in the art.

Second aspect of the present application relates to a process for preparing linaclotide by treating linear chain of peptide of formula (I) with dimethyl sulfoxide (DMSO) in water. The reaction between the linear chains of peptide of formula (I) with dimethyl sulfoxide (DMSO) in water may be performed at basic pH. Specifically, the reaction between the linear chains of peptide of formula (I) with dimethyl sulfoxide (DMSO) in water may be performed at a pH from about 8.0 to about 10.0. The ratio of water and DMSO in the reaction medium may be in the range of about 100:0.5 to about 100:10.0. Specifically, the ratio of water and DMSO in the reaction medium may be in the range of about 100:1.0 to about 100:5.0. More specifically, the ratio of water and DMSO in the reaction medium may be about 99:1.0. Optionally, a buffer such as ammonium sulfate may be added to the reaction mass to ensure that the pH of the reaction mass remains constant throughout the reaction. The reaction may be performed at about 15° C. to about 50° C. for about 1 hour to about 48 hours. Specifically, the reaction may be performed at about 20 to about 30° C. for about 15 hours to about 30 hours. The reaction may be worked-up by acidification of the reaction medium by a suitable acid such as trifluoroacetic acid, acetic acid and the product may be isolated by any known methods in the art.

Third aspect of the present application relates to a process for preparing linaclotide by treating linear chain of peptide of formula (I) with a complex of pyridine-sulfur trioxide. The reaction between linear chains of peptide of formula (I) with pyridine-sulfur trioxide may be performed in an aqueous solution in basic pH. Specifically, the reaction may be performed in water at a pH from about 8.0 to about 10.0. The reaction may be performed at about 15° C. to about 50° C. for about 1 hour to about 48 hours. Specifically, the reaction may be performed at about 20 to about 30° C. for about 15 hours to about 30 hours. The reaction may be worked-up by acidification of the reaction medium by a suitable acid such as trifluoroacetic acid, acetic acid and the product may be isolated by any known methods in the art.

Fourth aspect of the present application relates to a process for preparing linaclotide by treating linear chain of peptide of formula (I) with guanidine hydrochloride. The reaction between linear chains of peptide of formula (I) with guanidine hydrochloride may be performed in an aqueous solution in basic pH. Specifically, the reaction may be performed in water at a pH from about 8.0 to about 9.0. Optionally, the reaction may be performed in presence of a buffer such as ammonium sulfate. Optionally, the reaction may be performed in presence of a co-oxidant. The co-oxidant may be a mixture of cysteine and cystine. The reaction may be performed at about 15° C. to about 50° C. for about 1 hour to about 48 hours. Specifically, the reaction may be performed at about 20° C. to about 30° C. for about 15 hours to about 30 hours. The reaction may be worked-up by acidification of the reaction medium by a suitable acid such as trifluoroacetic acid, acetic acid and the product may be isolated by any known methods in the art.

Fifth aspect of the present application relates to a process for preparing linaclotide by treating linear chain of peptide of formula (I) with clear-OX™. The reaction between linear chains of peptide of formula (I) with clear-OX™ may be performed in an aqueous solution in basic pH. Specifically, the reaction may be performed in water at a pH from about 8.0 to about 9.0. Optionally, the reaction may be performed in presence of a buffer such as ammonium sulfate. The reaction may be performed at about 15° C. to about 50° C. for about 1 hour to about 10 hours. Specifically, the reaction may be performed at about 20° C. to about 30° C. for about 2 hours to about 5 hours. The reaction may be worked-up by acidification of the reaction medium by a suitable acid such as trifluoroacetic acid, acetic acid and the product may be isolated by any known methods in the art.

Sixth aspect of the present application relates to a process for preparing linaclotide by treating linear chain of peptide of formula (I) with reduced glutathione. The reaction between linear chains of peptide of formula (I) with reduced glutathione may be performed in an aqueous solution in basic pH. Specifically, the reaction may be performed in water at a pH from about 8.0 to about 9.0. Optionally, the reaction may be performed in presence of a buffer such as ammonium sulfate. The reaction may be performed at about 15° C. to about 50° C. for about 1 hour to about 10 hours. Specifically, the reaction may be performed at about 20° C. to about 30° C. for about 2 hours to about 5 hours.

Seventh aspect of the present application relates to a process for preparing linaclotide by treating linear chain of peptide of formula (I) with continuous supply of air in presence of dimethyl sulfoxide (DMSO). The reaction between linear chains of peptide of formula (I) with continuous supply of air in presence of DMSO may be performed in an aqueous solution in basic pH. Specifically, the reaction may be performed in a mixture of water and DMSO at a pH from about 8.0 to about 10.0. Optionally, the reaction may be performed in presence of a buffer such as ammonium sulfate. The reaction may be performed at about 15° C. to about 50° C. for about 1 hour to about 48 hours. Specifically, the reaction may be performed at about 20° C. to about 30° C. for about 15 hours to about 30 hours. The reaction may be worked-up by acidification of the reaction medium by a suitable acid such as trifluoroacetic acid, acetic acid and the product may be isolated by any known methods in the art.

Eighth aspect of the present application relates to a process for preparing linaclotide by treating linear chain of peptide of formula (I) with solid supported (2,2,6,6-tetramethylpiperidinyl-1-yl)oxy (TEMPO) in presence of a co-oxidant. The reaction between linear chains of peptide of formula (I) with solid supported TEMPO may be performed in aqueous solution in presence of a co-oxidant. Specifically, the reaction may be performed in water. Any co-oxidant known in the art may be used for the reaction. Specifically, the co-oxidant may be sodium hypochlorite. The reaction may be performed at about 15° C. to about 50° C. for about 1 hour to about 48 hours. Specifically, the reaction may be performed at about 20° C. to about 30° C. for about 15 hours to about 30 hours.

Ninth aspect of the present application relates to a process for preparing linaclotide by treating linear chain of peptide of formula (I) with water, without any oxidant. The reaction between linear chains of peptide of formula (I) with water, without any oxidant (such as DMSO) may be performed at a pH from about 8.0 to about 10.0. Optionally, the reaction may be performed in presence of a buffer such as ammonium sulfate. The reaction may be performed at about 15° C. to about 50° C. for about 1 hour to about 48 hours. Specifically, the reaction may be performed at about 20° C. to about 30° C. for about 15 hours to about 30 hours.

Tenth aspect of the present application relates to a process for preparing linaclotide by treating linear chain of peptide of formula (I) with hydrogen peroxide. The reaction between linear chains of peptide of formula (I) with hydrogen peroxide may be performed in an aqueous solution in basic pH. Specifically, the reaction may be performed in water at a pH from about 8.0 to about 10.0. Optionally, the reaction may be performed in presence of a buffer such as ammonium sulfate. The reaction may be performed at about 15° C. to about 50° C. for about 1 hour to about 48 hours. Specifically, the reaction may be performed at about 20° C. to about 30° C. for about 15 hours to about 30 hours.

Eleventh aspect of the present application relates to a process for preparing linaclotide by treating linear chain of peptide of formula (I) with potassium ferricyanide. The reaction between linear chains of peptide of formula (I) with potassium ferricyanide may be performed in an aqueous solution in basic pH. Specifically, the reaction may be performed in water at a pH from about 8.0 to about 10.0. The reaction may be performed at about 15° C. to about 50° C. for about 1 hour to about 48 hours. Specifically, the reaction may be performed at about 20° C. to about 30° C. for about 15 hours to about 30 hours.

Twelfth aspect of the present application relates to a process for preparing linaclotide by treating linear chain of peptide of formula (I) with manganese oxide. The reaction between linear chains of peptide of formula (I) with manganese oxide may be performed in an aqueous solution in basic pH. Specifically, the reaction may be performed in water at a pH from about 8.0 to about 9.0. The reaction may be performed at about 15° C. to about 50° C. for about 1 hour to about 48 hours. Specifically, the reaction may be performed at about 20° C. to about 30° C. for about 15 hours to about 30 hours.

Thirteenth aspect of the present application relates to a process for preparing linaclotide by treating linear chain of peptide of formula (I) with montmorillonite K-10. The reaction between linear chains of peptide of formula (I) with montmorillonite K-10 may be performed in an aqueous solution in basic pH. Specifically, the reaction may be performed in water at a pH from about 8.0 to about 9.0. The reaction may be performed at about 15° C. to about 50° C. for about 1 hour to about 48 hours. Specifically, the reaction may be performed at about 20° C. to about 30° C. for about 15 hours to about 30 hours.

Fourteenth aspect of the present application relates to a process for preparing linaclotide by treating linear chain of peptide of formula (I) with trimethylamine sulfur trioxide. The reaction between linear chains of peptide of formula (I) with trimethylamine sulfur trioxide may be performed in an aqueous solution in basic pH. Specifically, the reaction may be performed in water at a pH from about 8.0 to about 9.0. The reaction may be performed at about 15° C. to about 50° C. for about 1 hour to about 48 hours. Specifically, the reaction may be performed at about 20° C. to about 30° C. for about 15 hours to about 30 hours.

Fifteenth aspect of the present application relates to a process for preparing linaclotide by treating linear chain of peptide of formula (I) with vanadium pentoxide. The reaction between linear chains of peptide of formula (I) with vanadium pentoxide may be performed in an aqueous solution in basic pH. Specifically, the reaction may be performed in water at a pH from about 8.0 to about 9.0. The reaction may be performed at about 15° C. to about 50° C. for about 1 hour to about 48 hours. Specifically, the reaction may be performed at about 20° C. to about 30° C. for about 15 hours to about 30 hours.

Sixteenth aspect of the present application relates to a process for preparing linaclotide by treating linear chain of peptide of formula (I) with cysteine-cystine. The reaction between linear chains of peptide of formula (I) with cysteine-cysteine may be performed in an aqueous solution in basic pH. Specifically, the reaction may be performed in water at a pH from about 7.5 to about 9.0. The reaction may be performed at about 0° C. to about 25° C. for about 1 hour to about 48 hours. Specifically, the reaction may be performed at about 2° C. to about 4° C. for about 15 hours to about 30 hours. Optionally, the reaction may be performed in presence of sodium chloride and/or L-arginine.

The process for formation of the sulfide bonds in linaclotide, as described in the present application, may be carried out in an aqueous solvent. The aqueous solvent may optionally comprise a suitable organic solvent. The organic solvent may include but not limited to ethers such as diethyl ether, tetrahydrofuran and the like; esters such as ethyl acetate, methyl acetate and the like; alcohols such as methanol, ethanol and the like; aliphatic hydrocarbons such as hexane, heptane and the like; aromatic hydrocarbons such as toluene, xylene and the like; chlorinated hydrocarbons such as chloroform, dichloromethane and the like; polar aprotic solvents such as dimethyl sulfoxide, dimethyl formamide and the like.

The process for formation of the sulfide bonds in linaclotide, as described in the present application, is a simple and cost-effective process. Also, the process provides linaclotide in sufficiently pure form which may be directly used for purification process to provide amorphous form of linaclotide.

Seventeenth aspect of the present application relates to a purification process for the preparation of amorphous form of linaclotide comprising ion-exchange chromatography.

The crude reaction mass, obtained from any one of the above described reaction conditions may be purified to afford amorphous linaclotide. The eighteenth aspect of the present application relates to a purification process of crude linaclotide or a reaction mixture containing linaclotide comprising ion-exchange chromatography. Optionally, the reaction mixture containing linaclotide, as produced by any one of the reaction conditions described above, may undergo desalting process before purification by ion-exchange chromatography. In one specific embodiment, crude linaclotide or a reaction mixture containing crude linaclotide may be purified by anion-exchange chromatography. In another specific embodiment, crude linaclotide or a reaction mixture containing crude linaclotide may be purified by strong cation-exchange chromatography.

The column which may be used for the anion-exchange chromatography may be any column known in the art. Specifically, Source™ 15Q (GE Healthcare) resin may be used in the Fineline™ column for the purification of crude linaclotide. The mobile phase may be a mixture of sodium phosphate buffer solution and sodium chloride solution. The flow rate of the mobile phase may be set from about 20 mL/min to about 50 mL/min. Specifically, the flow rate may be set from about 25 mL/min to about 40 mL/min. The collected fractions may be analyzed by HPLC. The column may be regenerated by desorbing the highly charged impurities with a sodium chloride solution.

The column which may be used for the strong cation-exchange chromatography may be any column known in the art. Specifically, Source™ 15S (GE Healthcare) resin may be used in the Fineline™ column for the purification of crude linaclotide. The column may be regenerated by desorbing the highly charged impurities with a sodium chloride solution.

Linaclotide having a purity of more than about 70% may be obtained by the above described ion-exchange chromatography. Specifically, linaclotide having a purity of more than about 75% may be obtained by the above described ion-exchange chromatography. More specifically, linaclotide having a purity of more than about 80% may be obtained by the above described ion-exchange chromatography.

Linaclotide purified by anion-exchange chromatography, as described above, may be purified further by another anion-exchange chromatography and/or reverse-phase chromatography.

The column which may be used for the second anion-exchange chromatography may be any column known in the art. Specifically, Capto adhere ImPress (GE Healthcare) may be used in the column for the purification of crude linaclotide. The flow rate of the mobile phase may be set from about 20 mL/min to about 50 mL/min. Specifically, the flow rate may be set from about 25 mL/min to about 40 mL/min. Linaclotide was loaded at a rate of about 25 g per liter of resin. The collected fractions may be analyzed by HPLC. The column may be regenerated by desorbing the highly charged impurities with a sodium chloride solution.

The column which may be used for the reverse-phase chromatography may be any known column in the art. In one embodiment, the column may be a Kromasil C18 column. In another embodiment, the column may be a Phenomenex Luna C18 (2) column.

Linaclotide purified by strong cation-exchange chromatography, as described above, may be purified further by another cation-exchange chromatography and/or reverse-phase chromatography.

The column which may be used for the second strong cation-exchange chromatography may be any column known in the art. Specifically, Source™ 15Q (GE Healthcare) resin may be used in the Fineline™ column.

The column which may be used for the reverse-phase chromatography may be any known column in the art. In one embodiment, the column may be a Kromasil C18 column. In another embodiment, the column may be a Phenomenex Luna C18 (2) column.

Nineteenth aspect of the present application relates to a purification process for the preparation of amorphous form of linaclotide comprising purification by hydrophobic interaction. Twentieth aspect of the present application relates to a purification process of crude linaclotide or a reaction mixture containing linaclotide comprising purification by hydrophobic interaction. Optionally, the reaction mixture containing linaclotide, as produced by any one of the reaction conditions described above, may undergo desalting process before purification by hydrophobic interaction. The column which may be used for the purification by hydrophobic interaction may be any column known in the art. In one embodiment, HP20SS media in Novasep column may be used for the purification of crude linaclotide. In another embodiment, Purolite media in Novasep column may be used for the purification of crude linaclotide. The collected fractions may be analyzed by HPLC.

Linaclotide purified by hydrophobic interaction, as described above, may be purified further by another hydrophobic interaction and/or reverse-phase chromatography.

The column which may be used for the reverse-phase chromatography may be any known column in the art. In one embodiment, the column may be a Kromasil C18 column. In another embodiment, the column may be a Phenomenex Luna C18 (2) column.

The pooled fraction containing pure linaclotide may be freeze-dried using dry ice in acetone or liquid nitrogen. After freezing, the sample is lyophilized using vacuum at 200 mT and condenser temperature −100° C.

One skilled in the art would understand that the above purification techniques may result into a salt of linaclotide, depending upon the choice of mobile phase. The linaclotide salt may be converted to linaclotide by loading the linaclotide salt into a column and washing with a suitable mobile phase.

The purification process of linaclotide, described in the present application, provides at least 95% pure linaclotide. Specifically the purification process of linaclotide, described in the present application, provides at least 97% pure linaclotide. More specifically the purification process of linaclotide, described in the present application, provides at least 98% pure linaclotide. Most specifically, the purification process of linaclotide, described in the present application, provides at least 99% pure linaclotide. The purification process, described in the present application, is a simple and cost-effective process.

The linear chain of peptide of formula (I) may be prepared by any known methods in the art. Specifically, the linear chain of peptide of formula (I) may be prepared by solid phase synthesis. More specifically, the linear chain of peptide of formula (I) may be prepared by the process as disclosed in Benitez et al. Peptide Science, 2010, Vol. 96, No. 1, 69-80.

Certain specific aspects and embodiments are further described by the following examples, being provided only for purposes of illustration, and the scope of the disclosure is not intended to be limited by the examples.

### EXAMPLES

### Example 1

### Preparation of Crude Linaclotide using Polyvinyl Polymer Bound Complex of Sulfur Trioxide-pyridine

The linear chain of peptide of formula (I) (0.1 g) and polyvinyl polymer bound complex of sulfur trioxide-pyridine (0.062 g) was charged in water (100 mL). The pH of the reaction mass was adjusted to 8.5 to 9 by addition of ammonium hydroxide. The reaction mass was stirred at 25° C. for 15 hours and trifluoroacetic acid (2 mL) was added to the reaction mass to adjust the pH up to 2-2.5. The reaction mass was stirred for 3 hours at the same temperature to afford crude linaclotide.

### HPLC Purity: 59.92%

### Example 2

### Preparation of Crude Linaclotide using DMSO in Water

The pH of water (100 mL) was adjusted to 9.1 by the addition of aqueous ammonia. DMSO (1 mL) and linear chain of peptide of formula (I) (100 mg) were charged. The reaction mass was stirred for 17 hours at 25° C. and acidified with trifluoroacetic acid to pH 1.9 and stirred for 8 hours at the same temperature to afford crude linaclotide.

### HPLC Purity: 57%

### Example 3

### Preparation of Crude Linaclotide using DMSO in water

The pH of water (1500 mL) was adjusted to 9 by the addition of aqueous ammonia. DMSO (15 mL) and linear chain of peptide of formula (I) (15 g) were charged. The reaction mass was stirred for 17 hours at 25° C. and acidified with acetic acid to pH 1.9 and stirred for 8 hours at the same temperature to obtain crude linaclotide.

### HPLC Purity: 46.02%

### Example 4

### Preparation of Crude Linaclotide in Water

To a mixture of water (1900 mL) and ammonium sulfate (26.4 g), ammonium hydroxide was added drop wise to adjust the pH up to 8.5. Linear chain of peptide of formula (I) (26.4 g) was added and the reaction mass was stirred for 8 hours at 25° C. Trifluoroacetic acid (20 mL) was added drop wise and the reaction mixture was stirred for 15 hours at 25° C. to afford crude linaclotide. HPLC Purity: 63.38%

### Example 5

### Preparation of Crude Linaclotide using a Complex of Pyridine-sulfur Trioxide

Linear chain of peptide of formula (I) (0.2 g) was added to water (250 mL) and the pH of the reaction mass was adjusted to 8.91 by the drop wise addition of aqueous ammonia. A complex of pyridine-sulfur trioxide (0.124 g) was added to the reaction mass and stirred for 16 hours at 25° C. Another lot of complex of pyridine-sulfur trioxide (0.124 g) was added to the reaction mass and stirred for 5 hours at 25° C. to afford crude linaclotide.

### Example 6

### Preparation of Crude Linaclotide using Guanidine Hydrochloride

To a solution of sodium bicarbonate (0.89 g) in water (100 mL), cysteine (0.363 g), cysteine (0.072 g) and guanidine hydrochloride (9.50 g) were charged. Acetonitrile (15 mL) and linear chain of peptide of formula (I) (0.1 g) was added to the reaction mass. The reaction mass was stirred for 3 hours at 25° C. and trifluoroacetic acid (2 mL) was added. The reaction mass was stirred for 18 hours at the same temperature. Another lot of trifluoroacetic acid (2 mL) was added to the reaction mass and stirred for 18 hours at the same temperature to afford crude linaclotide.

### Example 7

### Preparation of Crude Linaclotide using Clear-OX™

Pre-conditioned Clear-Ox™ (0.5 g) was added to a solution of ammonium sulfate (1.32 g) in water (100 mL) of pH 8.5, adjusted by addition of ammonium hydroxide. The linear chain of peptide of formula (I) (0.1 g) was added to the reaction mass and stirred for 3 hours at 25° C. Another lot of Pre-conditioned Clear-Ox™ (0.5 g) was added to the reaction mass and stirred for 1.30 hours. Trifluoroacetic acid (2 mL) was added to the reaction mass and stirred for 16 hours at the same temperature to afford crude linaclotide.

### HPLC Purity: 67.5%

### Example 8

### Preparation of Crude Linaclotide using reduced Glutathione

To a mixture of ammonium sulphate (5.28 g) in water (400 mL) and isopropyl alcohol (400 mL), reduced glutathione (0.248 g) was added and the pH was adjusted to 8.5 by using aqueous ammonia. The linear chain of peptide of formula (I) (0.81 g) was added to the reaction mixture and stirred at ambient temperature for 17 hours. Isopropyl alcohol was evaporated under vacuum to afford crude linaclotide.

### HPLC Purity: 69.56%%

### Example 9

### Preparation of Crude Linaclotide using DMSO and Air Bubbling

To a mixture of water (95 mL) and ammonium sulfate (1.32 g), ammonium hydroxide was added drop wise to adjust the pH up to 8.5. Linear chain of peptide of formula (I) (0.1 g) and DMSO (5 mL) was added and the reaction mass was stirred for 20 hours at 25° C. with continuous air bubbling. Trifluoroacetic acid (2 mL) was added to the reaction mass and stirred for 19 hours with continuous air bubbling at the same temperature to afford the title product.

### HPLC Purity: 59.11%

### Example 10

### Preparation of Crude Linaclotide using Solid Supported TEMPO

To a mixture of water (100 mL) and silica bound TEMPO (0.01 g), linear chain of peptide of formula (I) (0.1 g) and sodium hypochlorite solution (1 mL) were added and the reaction mass was stirred 18 hours at 25° C. Another lot of sodium hypochlorite solution (0.5 mL) was added to the reaction mass and stirred for further 7 hours at the same temperature to afford title product.

### HPLC Purity: 42.70%

### Example 11

### Preparation of Crude Linaclotide using Air

The pH of water (1 L) was adjusted to 9 by the addition of aqueous ammonia and linear chain of peptide of formula (I) (1 g) was added to the reaction mass. The reaction mass was stirred at 25° C. for 17 hours with continuous air bubbling.

### HPLC Purity: 29.43%

### Example 12

### Preparation of Crude Linaclotide using Hydrogen Peroxide

The pH of water (100 mL) was adjusted to 9.57 by the addition of aqueous ammonia and linear chain of peptide of formula (I) (0.1 g) and hydrogen peroxide (0.3 mL) were added to the reaction mass at 25-35° C. The reaction mass was stirred for 23 hours at 25° C. Another lot of hydrogen peroxide (0.3 mL) was added to the reaction mass and stirred at 25° C. for further 46 hours to afford crude linaclotide.

### Example 13

### Preparation of Crude Linaclotide using Potassium Ferricyanide

The pH of water (500 mL) was adjusted to 9.54 by the addition of aqueous ammonia and linear chain of peptide of formula (I) (0.5 g) and potassium ferricyanide (0.1 g) were added to the reaction mass at 25° C. The reaction mass was stirred for 22 hours at 25° C. Another lot of potassium ferricyanide (0.1 g) was added to the reaction mass and stirred at 25° C. for further 2 hours to afford crude linaclotide.

### Example 14

### Preparation of Crude Linaclotide using Manganese Dioxide

The pH of water (20 mL) was adjusted to 8.5 by the addition of aqueous ammonia and linear chain of peptide of formula (I) (0.2 g) and manganese dioxide (4 mg) was added to the reaction mass at 25° C. The reaction mass was stirred for 18 hours at 25° C. to afford crude linaclotide.

### HPLC Purity: 42.3%

### Example 15

### Preparation of Crude Linaclotide using Montmorillonite K-10

The pH of water (20 mL) was adjusted to 8.5 by the addition of aqueous ammonia and linear chain of peptide of formula (I) (0.2 g) and montmorillonite K-10 (4 mg) was added to the reaction mass at 25° C. The reaction mass was stirred for 18 hours at 25° C. to afford crude linaclotide.

### HPLC Purity: 35-40%

### Example 16

### Preparation of Crude Linaclotide using Triethylamine-sulfur Trioxide

The pH of water (20 mL) was adjusted to 8.5 by the addition of aqueous ammonia and linear chain of peptide of formula (I) (0.2 g) and triethylamine-sulfur trioxide (4 mg) was added to the reaction mass at 25° C. The reaction mass was stirred for 18 hours at 25° C. to afford crude linaclotide.

### HPLC Purity: 33.9%

### Example 17

### Preparation of Crude Linaclotide using vanadium pentoxide

The pH of water (20 mL) was adjusted to 8.5 by the addition of aqueous ammonia and linear chain of peptide of formula (I) (0.2 g) and vanadium pentoxide (4 mg) was added to the reaction mass at 25° C. The reaction mass was stirred for 18 hours at 25° C. to afford crude linaclotide.

### Example 18

### Preparation of Crude Linaclotide using Cysteine and Cystine

The pH of a mixture of Tris(hydroxymethyl)aminomethane hydrochloride (50 mM), sodium chloride (500 mM), L-arginine (500 mM), cysteine (1 mM) and cystine (0.7 mM) in water was adjusted to 8 by the addition of aqueous hydrochloric acid and linear chain of peptide of formula (I) (2.0 g) was added to the reaction mass at 2-4° C. after bubbling the reaction mass with nitrogen for 2.0 hours. The reaction mass was stirred for 18-24 hours at 2-4° C. to afford crude linaclotide.

### Example 19

### Preparation of Crude Linaclotide using Cysteine and Cystine

The pH of a mixture of Tris(hydroxymethyl)aminomethane hydrochloride (50 mM), cysteine (1 mM) and cystine (0.7 mM) in water was adjusted to 8 by the addition of aqueous hydrochloric acid and linear chain of peptide of formula (I) (2.0 g) was added to the reaction mass at 2-4° C. after bubbling the reaction mass with nitrogen for 2.0 hours. The reaction mass was stirred for 18-24 hours at 2-4° C. to afford crude linaclotide.

### Example 20

### Purification of Crude Linaclotide comprising Anion-exchange Chromatography

The following column conditions were used for the purification of a solution of crude linaclotide in water by anion-exchange chromatography:
Column: Fine Line 35 column packed with Source 15Q, AKTA explorer
Mobile Phase A: Water
Mobile Phase B: 25 mM sodium phosphate buffer+0.1 M sodium chloride
Mobile Phase C: 2M sodium chloride
Gradient (CV/%13/%C): 1/2.5/0, 1/10/0, 2/20/0, 3.5/50/0, 3.5/100/0, 2/0/2
Flow Rate: 30-35 mL/min
Detection: 220 nm, 280 nm
Run Time: 180 min.
Sample Concentration: 1 mg/mL
Injection Volume: 7500 mL

The column was equilibrated with 2.5% of mobile phase B. The solution containing crude linaclotide was acidified with dilute acetic acid solution to pH 6.4 before loading. The column feed was then rinsed with 2.5% of mobile phase B and washed with 10% and 20% of mobile phase B, which removed the weakly charged impurities. Pure linaclotide was then eluted with 50% and 100% of mobile phase B. The collected fractions were analyzed by HPLC and the fractions containing more than about 80% were pooled together. The column was then regenerated with mobile phase C.

Pure linaclotide, as obtained by the above anion-exchange chromatography was purified further by another anion exchange chromatography.

The chromatographic condition was:
Column: Fine Line 35 column packed with Capto adhere ImPress, AKTA explorer
Mobile Phase A: Water
Mobile Phase B: 5% acetic acid
Mobile Phase C: 2M sodium chloride
Gradient (CV/%13/%C): 1/2.5/0, 1/10/0, 2/20/0, 3.5/50/0, 3.5/100/0, 2/0/2
Flow Rate: 30-35 mL/min

### Detection: 220 nm, 280 nm

Run Time: 180 min.
Sample Concentration: 1 mg/mL
Injection Volume: 7500 mL

The column was equilibrated with 2.5% of mobile phase B. The column feed was then rinsed with 2.5% of mobile phase B and washed with 10% and 20% of mobile phase B, which removed the weakly charged impurities. Pure linaclotide was then eluted with 50% and 100% of mobile phase B. The collected fractions were analyzed by HPLC and the fractions containing more than about 98% were pooled together. The column was then regenerated with mobile phase C.

Pure linaclotide, as obtained by the above anion-exchange chromatography, was purified further by reverse-phase chromatography (RP-HPLC). The chromatographic condition was:
Column: Kromasil 100 C18, 10 μm
Mobile Phase A: 0.01 M ammonium acetate in water, pH adjusted to 4.3 with acetic acid
Mobile Phase B: Acetonitrile
Gradient (T/%B): 0/5, 30/25, 39/25, 40/100, 50/100
Flow Rate: 30 mL/min
Detection: 220 nm
Run Time: 50 min.
Diluent: Water: Acetonitrile: Acetic acid (1:1:1)
Sample Concentration: 50 mg/mL
Injection Volume: 5 mL
Retention Time: 23.6 to 25.6 min.

The pooled fractions having HPLC purity of more than about 98% was collected and lyophilized to afford amorphous linaclotide.

### Example 21

### Purification of Crude Linaclotide Comprising Strong Cation-exchange Chromatography

The following column conditions were used for the purification of a solution of crude linaclotide in water by strong cation-exchange chromatography:

### Column: Fineline™ 100 mm

Media: Source™ 15S
Length: 25 cm
Internal diameter: 100 mm
Column Pressure: 20 bar
Mobile phase A: 0.15% (v/v) orthophosphoric acid pH adjusted up to 2.2 with triethylamine
Mobile phase B: 0.15% (v/v) orthophosphoric acid pH adjusted up to 4.7 with triethylamine
Mobile phase C: 0.15% (v/v) orthophosphoric acid pH adjusted up to 6.0 with triethylamine (v/v).
Gradient: 1. Equilibrating with mobile phase A (2 CV)
   2. Sample loading
   3. Unbound wash with mobile phase A (2 CV)
   4. Gradient 0-20% in 15 min with mobile phase B 80.0 mL/min
   5. Gradient 20-40% in 15 min with mobile phase B 80.0 mL/min
   6. Gradient 40-60% in 15 min with mobile phase B 80.0 mL/min
   7. Gradient 60-80% in 15 min with mobile phase B 80.0 mL/min
   8. Gradient 80-95% in 15 min with mobile phase B 80.0 mL/min
   9. 95% isocretic with mobile phase B 80.0 mL/min
   10. Washed with pH 6.0 buffer till multimer eluted
Detection: 220 nm
Retention time: 450 to 600 min
Sample loading: 70 mL/min
Fraction collection: The target fraction from Retention time about 450-600 were collected manually at every 1.0 L solution.
   The pH of a solution of crude linaclotide (11,000 mL) was adjusted to 2.2 with orthophosphoric acid and/or triethylamine and filtered through 0.25μ filter. It was loaded in to the Fineline™ column having Source™ 15S (GE Healthcare) resin with a flow rate of 70 mL/min. After loading the sample, linaclotide was eluted by using 200 mL/min flow rate with 0.2% (v/v) trifluoroacetic acid in water as mobile phase A and 0.2% (v/v) trifluoro acetic acid in acetonitrile as mobile phase B with a linear gradient and with UV detection at 220 nm. The product fraction was collected manually. The collected fractions were analyzed and pooled. The fractions above 85% purity and multimer below 6.0% are pooled. Acetonitrile was removed from the pooled fractions.

Linaclotide, as obtained by the above strong cation-exchange chromatography was purified further by another strong cation-exchange chromatography. The chromatographic condition was same as above. The fractions above 90% purity and multimer below 3.0% are pooled.

Linaclotide, as obtained by the above strong cation-exchange chromatography, was purified further by reverse-phase chromatography (RP-HPLC). The chromatographic condition was:
Column: Phenomenex Luna C18 (2)
Media: Phenomenex C-18 Media (10μ)
Length: 25 cm
Internal diameter: 50 mm
Column Pressure: 65 bar
Mobile phase A: 0.05% (v/v) trifluoroacetic acid in water
Mobile phase B: 0.05% (v/v) trifluoroacetic acid in acetonitrile

Gradient table

| | | | Flow rate |
|---|---|---|---|
| Time | Mobile phase A | Mobile phase B | (mL/min) |
| 0 | 95 | 5 | 40 |
| 20 | 82 | 18 | 40 |
| 30 | 82 | 18 | 40 |
| 60 | 80 | 20 | 40 |
| 90 | 80 | 20 | 40 |
| 100 | 20 | 80 | 40 |
| 110 | 20 | 80 | 40 |

Detection: 220 nm
Run time: 110 min
Retention time: 62 to 65 min
Sample loading: 50 mL/min loading
Fraction collection: The target fraction from retention time 62-65 min were collected manually at every 3 minute

The solution of pure linaclotide, as obtained from strong cation-exchange chromatography, as described above was loaded on to C18 column (10μ) with a flow rate of 50 mL/min. Pure linaclotide was eluted by using 40 mL/min flow rate with 0.05% (v/v) trifluoroacetic acid in water as mobile phase A and 0.05% trifluoroacetic acid in acetonitrile as mobile phase B with a linear gradient and with UV detection at 220 nm. The product fraction is collected manually. The collected fractions are analyzed and pooled and distilled to remove Acetonitrile. After each injection time, the column was washed with buffer (50:50) for 20 min and then with buffer (95:5) for 30 mins to load the next sample on the column. The fractions collected were analyzed for purity by analytical HPLC and the fractions above 99.0% purity, single maximum impurity below 0.5% and Multimer below 0.5% were pooled.

The collected fraction was freezed by using dry ice in acetone and lyophilized by using vacuum at 200 mT and condenser temperature −100° C. to afford pure amorphous trifluoroacetic acid salt of linaclotide.
Yield: 50%
Purity (by HPLC): 99.07%

### Example 22

### Purification of Crude Linaclotide comprising Hydrophobic Interaction

The following column conditions were used for the purification of a solution of crude linaclotide in water by hydrophobic interaction:
Column: Novasep 80 mm column packer
Media: HP20SS Media (100 μ)
Length: 28 cm
Internal diameter: 80 mm
Column Pressure: 35 bar-40 bar (While packing)
Pressure gauge: 2.0 Mpa (While packing)
Mobile phase A: 0.2% (v/v) trifluoroacetic acid in water
Mobile phase B: 0.2% (v/v) trifluoroacetic acid in acetonitrile

Gradient table:

| | | | Flow rate |
|---|---|---|---|
| Time | Mobile phase A | Mobile phase B | (mL/min) |
| 0 | 100 | 0 | 200 |
| 7 | 100 | 0 | 200 |
| 10 | 95 | 5 | 200 |
| 15 | 90 | 10 | 200 |
| 40 | 80 | 20 | 200 |
| 80 | 80 | 20 | 200 |
| 95 | 65 | 35 | 200 |
| 100 | 0 | 100 | 200 |
| 120 | 0 | 100 | 200 |

Detection: 220 nm

Run time: 120 min

Retention time: 55 to 76 min

Sample loading: 200 mL/min

Fraction collection: The target fraction from Retention time about 55-76 were collected manually at every 3 minutes

Fraction pooling: The fractions collected were analyzed for purity by analytical HPLC and the fractions above 80% purity and multimer below 8.0% are pooled.

The pH of a solution of crude linaclotide (8,000 mL) was adjusted to 6.0 with trifluoroacetic acid and/or triethylamine and loaded in to in to the HP20SS Media in Novasep Column with a flow rate of 200 mL/min. After loading the sample, the unbound material in the column was washed with 0.15% (v/v) orthophosphoric acid having a pH adjusted up to 2.2 with triethylamine (2 CV) then pure Linaclotide was eluted with 0.15% (v/v) orthophosphoric acid having a pH adjusted up to 4.7 with triethylamine with a linear gradient, with UV detection at 220 nm and with flow rate of 80 mL/min. The product fraction was collected manually. The collected fractions were analyzed and pooled. The fractions above 80% purity and multimer below 8.0% were pooled. After each injection time, the column was washed with mobile phase B for 20 min and then with mobile phase A for 40 min to reactivate the column.

Linaclotide, as obtained by the above hydrophobic interaction was purified further by another hydrophobic interaction. The chromatographic condition was same as above. The fractions above 90% purity and multimer below 3.0% were pooled.

Linaclotide, as obtained by the above strong hydrophobic interaction, was purified further by reverse-phase chromatography (RP-HPLC). The chromatographic condition was:
Column: Phenomenex Luna C18 (2)
Media: Phenomenex C-18 Media, (10μ)
Length: 25 cm
Internal diameter: 50 mm
Column Pressure: 65 bar
Mobile phase A: 0.05% (v/v) trifluoroacetic acid in water
Mobile phase B: 0.05% (v/v) trifluoroacetic acid in acetonitrile

Gradient table

| | | | Flow rate |
|---|---|---|---|
| Time | Mobile phase A | Mobile phase B | (mL/min) |
| 0 | 95 | 5 | 40 |
| 20 | 82 | 18 | 40 |
| 30 | 82 | 18 | 40 |
| 60 | 80 | 20 | 40 |
| 90 | 80 | 20 | 40 |
| 100 | 20 | 80 | 40 |
| 110 | 20 | 80 | 40 |

Detection: 220 nm
Run time: 110 min
Retention time: 62 to 65 min
Sample loading: 50 mL/min
Fraction collection: The target fraction from Retention time 62-65 were collected manually at every 3 minute
   The fractions collected were analyzed for purity by analytical HPLC and the fractions above 99.0% purity, single maximum impurity below 0.5% and multimer below 0.5% were pooled and lyophilized.
   The lyophilized material, as obtained, was purified by further RP-HPLC for removing trifluoroacetic acid by following chromatographic conditions:
Column: Phenomenex Luna C18 (2)
Media: Phenomenex C-18 Media, (10μ)
Length: 25 cm
Internal diameter: 50 mm
Column Pressure: 65 bar
Mobile phase A: Water
Mobile phase B: Acetonitrile

Gradient table

| | | | Flow rate |
|---|---|---|---|
| Time | Mobile phase A | Mobile phase B | (mL/min) |
| 0 | 100 | 0 | 40 |
| 20 | 100 | 0 | 40 |
| 35 | 80 | 20 | 40 |
| 40 | 80 | 20 | 40 |
| 50 | 20 | 80 | 40 |
| 60 | 20 | 80 | 40 |

Detection: 220 nm
Run time: 60 min
Retention time: 14 to 18 min
Sample loading: 50 mL/min
   The collected fraction was freezed by using dry ice in acetone and lyophilized by using vacuum at 200mT and condenser temperature -100° C. to afford pure amorphous linaclotide.
Yield: 49%
Purity (by HPLC): 99.6%

1. A process for preparing linaclotide by treating a linear chain of peptide of formula (I) with a suitable reagent to prepare appropriate disulfide bridges within linear chain of peptide of formula (I)
Formula (I);
1 2 3 4 5 6 7 8 9 10 11 12 13 14
H-Cys-Cys-Glu-Tyr-Cys-Cys-Asn-Pro-Ala-Cys-Thr-Gly-Cys-Tyr-OH
wherein, the suitable reagent is selected from the group consisting of polymer bound complex of sulfur trioxide-pyridine, dimethyl sulfoxide (DMSO) in water, a complex of pyridine-sulfur trioxide, guanidine hydrochloride, clear-OX™, reduced glutathione, air in presence of DMSO, solid supported (2,2,6,6-tetramethylpiperidinyl-1-yl)oxy (TEMPO) in presence of a co-oxidant, in water without any oxidant, hydrogen peroxide, potassium ferricyanide, manganese oxide, montmorillonite K-10, trimethylamine sulfur trioxide, vanadium pentoxide and cysteine-cystine.

2. The process of claim 1, wherein linaclotide is prepared by treating a linear chain of peptide of formula (I) in an aqueous solvent.

3. The process of claim 2, wherein the aqueous solvent optionally comprises an organic solvent.

4. The process of claim 1, wherein linaclotide is prepared by treating a linear chain of peptide of formula (I) with 1% dimethyl sulfoxide (DMSO) in water.

5. The process of claim 1, wherein linaclotide is prepared by treating a linear chain of peptide of formula (I) with 1% dimethyl sulfoxide (DMSO) in water at pH from about 8 to about 10.

6. The process of claim 4, wherein the temperature is about 20° C. to about 30° C.

7. The process of claim 4, wherein the reaction time is from about 15 hours to about 30 hours.

8. A purification process for the preparation of amorphous form of linaclotide comprising ion-exchange chromatography.

9. The purification process of claim 8, wherein amorphous form of linaclotide is prepared comprising anion-exchange chromatography.

10. The purification process of claim 8, further comprises another anion-exchange chromatography and/or reverse-phase chromatography.

11. The purification process of claim 8, wherein amorphous form of linaclotide is prepared comprising strong cation-exchange chromatography.

12. The purification process of claim 8, further comprises another strong cation-exchange chromatography and/or reverse-phase chromatography.

13. A purification process for the preparation of amorphous form of linaclotide comprising hydrophobic interaction.

14. The purification process of claim 13, further comprises another hydrophobic interaction and/or reverse-phase chromatography.


SEQUENCE LISTING (1 sequence)

SEQ ID NO: 1 - protein, 14 aa, Artificial Sequence
  Synthetically generated peptide
CCEYCCNPACTGCY